(19) United States
(12) Patent Application Publication
(10) Pub. No.: US 2024/0009124 A1
(43) Pub. Date: Jan. 11, 2024

(54) Ophthalmic Suspension Base having a Micro-fluidized Positively Charged Nanoparticle

(71) Applicant: Primera Compounding, LLC, Rosenberg, TX (US)

(72) Inventors: Paula Hooper, Richmond, TX (US); Paramita Sarkar, Richmond, TX (US)

(21) Appl. No.: 18/345,674

(22) Filed: Jun. 30, 2023

Related U.S. Application Data
(60) Provisional application No. 63/358,410, filed on Jul. 5, 2022.

(51) Int. Cl. A61K 9/107; A61K 47/26; A61K 45/06; A61K 47/38; A61K 47/18

(52) U.S. Cl. CPC A61K 9/1075; A61K 47/26; A61K 45/06; A61K 47/38; A61K 47/186; B82Y 5/00

(57) ABSTRACT

An ophthalmic suspension contains a micro-fluidized positively charged nanoparticle incorporated into a viscous hydroxypropyl methylcellulose (HPMC) base. Positive charges on the surface of the nanoparticle ensure dispersion within solution while the viscosity prolongs the contact time with the ocular surface.

### CROSS REFERENCE TO RELATED APPLICATIONS

This application claims priority to U.S. Provisional Patent Application Ser. No. 63/358,410 filed on Jul. 5, 2022. The entire disclosure of that patent application is part of the disclosure of the present application and is hereby incorporated by reference in its entirety.

### FIELD OF THE INVENTION

The present invention relates to an ophthalmic dispersion for treating a broad range of ocular conditions. In particular, the present invention discloses an ophthalmic dispersion that includes a positively charged nanoparticle.

### BACKGROUND

The eye is a complex and delicate organ consisting of two primary anatomical portions. Most familiar is the anterior portion containing the tissues located at or near the surface of the eye. These tissues include the cornea, conjunctiva, iris, lens, and capillary body. Many diseases and disorders occur in this region such as dry eye, cataracts, bacterial infections, glaucoma, tumors, injury, and other ophthalmic conditions. It is common and straightforward from a clinical perspective to treat conditions of the anterior portion of the eye with drops. This mode of treatment has advantages over other treatments, such as oral or intravenous administration, because the therapy is localized to the ocular tissue. Furthermore, the accessibility of the eye surface permits patients to administer the eye drops on their own.

Although eye drops are easy to use and preferable over other modes of administration, current formulations suffer from poor efficacy. The eye has several barriers at the surface to protect against environmental perturbations: tear film, corneal, conjunctival, blood aqueous, and blood retinal barriers. For a topical treatment to be effective, eye drops must promote the prolongation of contact time at the surface and enhance permeability into the ocular tissue.

The inclusion of nanoparticles enables a significant improvement in eye drop formulation toward the treatment of ophthalmic conditions. Nanoparticles have been shown to increase permeability, stability, and extend the contact time at the ocular surface. The design of nanoparticle containing therapies is, however, a challenging endeavor.

### SUMMARY

In one embodiment, the present invention comprises an ophthalmic suspension base consisting essentially of: a plurality of positively charged nanoparticles incorporated in a viscous cellulose ether, wherein each of the nanoparticles is about 50 nm to 200 nm in diameter.

In another embodiment, the present invention comprises an ophthalmic suspension base consisting essentially of: a plurality of positively charged nanoparticles incorporated in a viscous cellulose ether and a pharmaceutically active agent, wherein each of the nanoparticles is about 50 nm to 200 nm in diameter.

The nanoparticles can include a nanoemulsion or liposomal nanoparticles.

The liposomal nanoparticle comprises a mixture of: a lecithin; a polysorbate; and a cation selected from the group consisting of a cetylpyridinium halide, a cetrimonium halide, a cetalkonium halide, or mixtures thereof. Sterile water can be included with the lecithin. The lecithin can be a complex mixture of soy derived phospholipids, glycolipids, and triglycerides having a concentration of about 0.1%-0.5% by weight of the total weight of the ophthalmic suspension base (w/w). The polysorbate can be polysorbate 80 having a concentration of about 0.1%-0.5% by weight of the total weight of the ophthalmic suspension base (w/w). The cetylpyridinium halide can be cetylpyridinium chloride having a concentration of about 0.002% by weight of the total weight of the ophthalmic suspension base (w/w). The cetrimonium halide is cetrimonium bromide. The cetalkonium halide can be cetalkonium chloride. The cellulose ether is hydroxypropyl methylcellulose (HPMC) having a concentration of about 0.2%-1.1% by weight of the total weight of the ophthalmic suspension base (w/w).

The nanoemulsion comprises a mixture of substantially equal amounts of heavy and light mineral oil having a concentration of about 0.5% by weight of the total weight of the ophthalmic suspension base (w/w), a lecithin; a polysorbate; and a cation selected from the group consisting of a cetylpyridinium halide, a cetrimonium halide, a cetalkonium halide, or mixtures thereof. Sterile water can be included with the lecithin. The lecithin can be a complex mixture of soy derived phospholipids, glycolipids, and triglycerides having a concentration of about 0.1%-0.5% by weight of the total weight of the ophthalmic suspension base (w/w). The polysorbate can be polysorbate 80 having a concentration of about 0.1%-0.5% by weight of the total weight of the ophthalmic suspension base (w/w). The cetylpyridinium halide can be cetylpyridinium chloride having a concentration of about 0.002% by weight of the total weight of the ophthalmic suspension base (w/w). The cetrimonium halide is cetrimonium bromide. The cetalkonium halide can be cetalkonium chloride. The cellulose ether is hydroxypropyl methylcellulose (HPMC) having a concentration of about 0.2%-1.1% by weight of the total weight of the ophthalmic suspension base (w/w).

The pH of the ophthalmic suspension base can be adjusted to around 6 to 8.

In another embodiment, the ophthalmic suspension base includes a pharmaceutically active agent. The pharmaceutically active agent can be an antibiotic, a nonsteroidal anti-inflammatory agent or any other desired agent. The pharmaceutically active agent has a concentration of about 0.1%-1.0% by weight of the total weight of the ophthalmic suspension base (w/w).

In another embodiment, a method for making the nanoemulsion ophthalmic suspension base, comprises preparing a biphasic mixture by: a) mixing a heavy and a light mineral oil, a polysorbate surfactant, and a cation selected from the group consisting of a cetylpyridinium halide, a cetrimonium halide, a cetalkonium halide, or mixtures thereof (“Phase A”); b) mixing lecithin and sterile water for injection (“Phase B”); c) mixing Phase A with Phase B to form an emulsion mixture; d) processing the emulsion through a microfluidizer to form a nanoemulsion; and e) incorporating the nanoemulsion into a cellulose ether. The method further involves adding a pharmaceutically active agent to the nanoemulsion and adjusting the pH of the nanoemulsion ophthalmic suspension base around 6 to 8.

In another embodiment, a method for making a nanoliposome ophthalmic suspension base, comprises: a) preparing a mixture of a polysorbate surfactant and a cation selected from the group consisting of a cetylpyridinium halide, a cetrimonium halide, a cetalkonium halide, or mixtures thereof; b) mixing lecithin and sterile water for injection; c) preparing a combined mixture of the polysorbate surfactant and cation mixture and the lecithin and sterile water mixture; d) processing the combined mixture through a microfluidizer to form a nanoliposome; and e) incorporating the nanoliposome into a cellulose ether. The method further involves adding a pharmaceutically active agent to the nanoemulsion and adjusting the pH of the nanoemulsion ophthalmic suspension base around 6 to 8.

### DETAILED DESCRIPTION

Eye drops are used to treat a myriad of problems associated with the ocular tissue. The present invention addresses a problem that has not been adequately solved in the prior art and is novel relative to the prior art. The present invention provides an eye drop composition or an ophthalmic suspension base containing a micro-fluidized positively charged nanoparticle incorporated into a viscous cellulose ether.

Each ingredient has been optimized for application to the eye. The positive charges on the surface of the nanoparticle ensure dispersion within solution while the viscosity prolongs the contact time with the ocular surface. The ophthalmic suspension base can be used as a treatment for dry eyes by itself and can serve as a moisturizer. Other embodiments of the ophthalmic suspension base can include pharmaceutically active agents or ingredients incorporated into the ophthalmic suspension base. When an active agent is incorporated within the positively charged nanoparticle, the agent can be delivered by the nanoparticle to the eye to treat the corresponding disease or disorder.

The term and phrases “invention,” “present invention,” “instant invention,” and similar terms and phrases as used herein are non-limiting and are not intended to limit the present subject matter to any single embodiment, but rather encompass all possible embodiments as described.

As used herein, all weight percentages (wt. %) are based on the total wt. % of the ophthalmic suspension base, unless otherwise specified. Additionally, all percentages are based on totals equal to 100 wt. %, unless otherwise specified.

The ophthalmic suspension base and methods for their use can “comprise,” “consist essentially of,” or “consist of” any of the ingredients or steps disclosed throughout the specification. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and can include the ingredients of the present invention and do not exclude other ingredients or elements described herein. The use of the word “a” or “an” when used in conjunction with the term “comprising” in the claims and/or the specification may mean “one,” but it is also consistent with the meaning of “one or more,” “at least one,” and “one or more than one.” As used herein, “consisting essentially of” means that the invention may include ingredients in addition to those recited in the claims, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the composition (as opposed to the degree of utility) is maintained.

All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. In one non-limiting embodiment, the terms are defined to be within 5%. The term “substantially” and its variations are defined as being largely but not necessarily wholly what is specified as understood by one of ordinary skill in the art, and in one non-limiting embodiment substantially refers to ranges within 0.01% to 5%.

The present invention relates to an ophthalmic suspension base or composition for treating a broad range of ocular conditions. The ophthalmic suspension base includes a plurality of positively charged nanosized particles made from either an emulsion or a liposome incorporated into a cellulose derived base and optional one or more pharmaceutically active agents. A liposome is an aqueous compartment enclosed by a bimolecular phospholipid membrane; while an emulsion is a stable suspension of small droplets of one liquid in another liquid with which it is immiscible and stabilized by an emulsifying agent.

The nanoemulsion is typically formed from a combination of mineral oil; water; a nonionic surfactant such as a polysorbate; a cation such as a cetylpyridium halide, a cetrimonium halide, a cetalkonium halide, or mixtures thereof; lecithin and sterile water for injection. The liposomal nanoparticle is formed from a combination of water; a non-ionic surfactant; a cation such as a cetylpyridium halide, a cetrimonium halide, a cetalkonium halide, or mixtures thereof; lecithin and sterile water for injection. The cationic compound is chosen so that the solvent exposed surface area of the nanoparticle carries a positive charge. Some embodiments of the ophthalmic suspension base use a lecithin that is a complex mixture of soy derived phospholipids, glycolipids, and triglycerides.

An embodiment of an ophthalmic suspension base is made as follows: a) mixing a heavy and a light mineral oil, a polysorbate surfactant, and a cation selected from the group consisting of a cetylpyridinium halide, a cetrimonium halide, a cetalkonium halide, or mixtures thereof (“Phase A”); b) mixing lecithin and water (“Phase B”); c) preparing a biphasic mixture/emulsion by mixing Phase A with Phase B; d) processing the emulsion through a microfluidizer to form nanoparticles from about 50 nm to 200 nm in diameter; e) preparing a cellulose derived base selected from the group consisting of a hydroxypropyl methylcellulose (HPMC), a microcrystalline cellulose, a hydroxyethylcellulose, or a methylcellulose, or mixtures thereof by mixing the cellulose derived base with propylene glycol and glycerin to prepare a cellulose base mixture and adjusting the pH the cellulose based mixture with sodium hydroxide or citric acid to a pH of 4.0 to 8.0; and F) adding the nanoemulsion to the cellulose derived base. In one exemplary embodiment, the cellulose derived base is a cellulose ether, such as HPMC.

One embodiment of a nanoemulsion is made as follows: step (a) mix equal amounts of heavy and light mineral oils (0.5% w/w) with a non-ionic surfactant such as polysorbate 80 (0.1% to 0.5% w/w), and a cation such as cetylpyridinium chloride (CPC) (0.02% w/w); step (b) in a separate container mix lecithin (0.1% to 0.5% w/w) with sterile water; step (c) mix the oil phase from step (a) with the lecithin phase from step (b); and step (d) process the mixed phases through a microfluidizer.

Another embodiment of the ophthalmic suspension base is made as follows: a) mixing a polysorbate surfactant and a cation selected from the group consisting of a cetylpyridinium halide, a cetrimonium halide, a cetalkonium halide, or mixtures thereof (“step 1”); b) mixing lecithin and water (“step 2”); c) combining the mixtures from step 1 and step 2; d) processing the combined mixture through a microfluidizer to form liposomal nanoparticles from about 50 nm to 200 nm in diameter; e) preparing a cellulose derived base selected from the group consisting of a hydroxypropyl methylcellulose, a microcrystalline cellulose, a hydroxyethylcellulose, or a methylcellulose, or mixtures thereof by mixing the cellulose base with propylene glycol and glycerin to prepare a cellulose base mixture and adjusting the pH the cellulose based mixture with sodium hydroxide or citric acid to a pH of 4.0 to 8.0; and f) adding the combined mixture to the cellulose derived base. In one exemplary embodiment, the cellulose derived base is a cellulose ether, such as HPMC.

One embodiment of a liposome is made as follows: step (a) mix a surfactant such as polysorbate 80 (0.1% to 0.5% w/w) and a cation such as cetylpyridinium chloride (0.02% w/w); step (b) in a separate container mix lecithin (0.1% to 0.5% w/w) with sterile water; step (c) mix the emulsifier and cation mixture from step (a) with the lecithin phase from step (b); and step (d) process the mixed phases through a microfluidizer.

Positive surface charges ensure that the nanoparticles are evenly dispersed through electrostatic repulsion. Other suitable cationic molecules can be employed, in combination with CPC or alone, such as cetrimonium bromide (CTAB) or cetalkonium chloride (CKC). The size of the nanoparticles is homogenous to further promote even dispersion in the composition. Following micro-fluidization, the size of the nanoparticles ranges from about 50 nm to 200 nm in diameter. Advantageously, the nanoparticles are carried by a base with variable viscosity. The viscosity enables the nanoparticles to linger on the surface of the eye for longer, thus increasing the chance for uptake. In one exemplary embodiment, the cellulose derived base is hydroxypropyl methylcellulose (HPMC) used at 0.2% to 1.1% w/w. In other embodiments of the ophthalmic suspension base, the cellulose derived base can be a cellulose derivative such as microcrystalline cellulose, hydroxyethylcellulose, or methylcellulose. The viscosity of the ophthalmic suspension base can be varied by manipulating the concentration of the cellulose derived base. Once the viscous base is prepared and buffered to pH 4 to 8, the microfluidized emulsion or liposome is added to it. The pH of this final composition is then adjusted with HCl or NaOH to between pH 6 and 8.

In exemplary embodiments, the ophthalmic suspension base includes at least one pharmaceutically active agent or drug. The active agent is carried by the nanoparticles to the ocular surface through electrostatic forces and physical interactions. Accordingly, the beneficial properties of the nanoparticles (i.e., even dispersion and increased absorption) are adopted by the active agent. The ophthalmic suspension base can include any active agent that is compatible with the nanoparticles and viscous base. It is anticipated that lipophilic, cationic, and neutral charge molecules will benefit from inclusion into the disclosed invention. One exemplary embodiment of a pharmaceutically active agent that may be carried by the composition is an antibiotic such as moxifloxacin HCl at 0.5% w/w or azithromycin dihydrate at 0.5 to 1.0% w/w. Another exemplary embodiment of a pharmaceutically active agent that may be carried by the composition is a nonsteroidal anti-inflammatory agent, such as, diclofenac sodium at 0.1% w/w or bromfenac sodium at 0.075% w/w.

The present invention is not limited to its use in the eyes and has other applications in which it may be particularly useful. A viscous base with a nanoparticle dispersion can also be advantageously applied intranasally or topically to the skin.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

1. An ophthalmic suspension base consisting essentially of:
a plurality of positively charged nanoparticles incorporated in a viscous cellulose derived base, wherein each of the nanoparticles is about 50 nm to 200 nm in diameter.

2. The ophthalmic suspension base according to claim 1, wherein the nanoparticle comprise a mixture of:
a lecithin;
a polysorbate; and
a cation selected from the group consisting of a cetylpyridinium halide, a cetrimonium halide, a cetalkonium halide, or mixtures thereof.

3. The ophthalmic suspension base according to claim 2, wherein the lecithin is a complex mixture of soy derived phospholipids, glycolipids, and triglycerides having a concentration of about 0.1%-0.5% by weight of the total weight of the ophthalmic suspension base (w/w).

4. The ophthalmic suspension base according to claim 2, wherein the polysorbate is polysorbate 80 having a concentration of about 0.1%-0.5% by weight of the total weight of the ophthalmic suspension base (w/w).

5. The ophthalmic suspension base according to claim 2, wherein the cetylpyridinium halide is cetylpyridinium chloride having a concentration of about 0.002% by weight of the total weight of the ophthalmic suspension base (w/w).

6. The ophthalmic suspension base according to claim 2, wherein the cetrimonium halide is cetrimonium bromide.

7. The ophthalmic suspension base according to claim 2, wherein the cetrimonium halide is cetrimonium bromide.

8. The ophthalmic suspension base according to claim 2, wherein the cetalkonium halide is cetalkonium chloride.

9. The ophthalmic suspension base according to claim 2, wherein the nanoparticles further comprise substantially equal amounts of heavy and light mineral oil having a concentration of about 0.5% by weight of the total weight of the ophthalmic suspension base (w/w).

10. The ophthalmic suspension base according to claim 1, wherein the cellulose derived base is a cellulose ether.

11. The ophthalmic suspension base according to claim 10, wherein the cellulose derived base is hydroxypropyl methylcellulose (HPMC) having a concentration of about 0.2%-1.1% by weight of the total weight of the ophthalmic suspension base (w/w).

12. The ophthalmic suspension base according to claim 1, wherein the nanoparticles comprise a nanoemulsion or liposomal nanoparticles.

13. The ophthalmic suspension base according to claim 1, wherein the pH of the ophthalmic suspension base is around 6 to 8.

14. An ophthalmic suspension base consisting essentially of:
a plurality of positively charged nanoparticles incorporated in a viscous cellulose ether and a pharmaceutically active agent, wherein each of the nanoparticles is about 50 nm to 200 nm in diameter.

15. The ophthalmic suspension base according to claim 14, wherein the pharmaceutically active agent is an antibiotic.

16. The ophthalmic suspension base according to claim 14, wherein the pharmaceutically active agent is a nonsteroidal anti-inflammatory agent.

17. The ophthalmic suspension base according to claim 14, wherein the pharmaceutically active agent has a concentration of about 0.1%-1.0% by weight of the total weight of the ophthalmic suspension base (w/w).

18. A method for making a nanoemulsion ophthalmic suspension base, comprising:
a) mixing a heavy and a light mineral oil, a polysorbate surfactant, and a cation selected from the group consisting of a cetylpyridinium halide, a cetrimonium halide, a cetalkonium halide, or mixtures thereof (“Phase A”);
b) mixing lecithin and sterile water for injection (“Phase B”);
c) mixing Phase A with Phase B to form an emulsion mixture;
d) processing the emulsion through a microfluidizer to form a nanoemulsion; and
e) incorporating the nanoemulsion into a cellulose ether.

19. The method according to claim 18, further comprising adding a pharmaceutically active agent to the nanoemulsion.

20. The method according to claim 18, further comprising adjusting the pH of the nanoemulsion ophthalmic suspension base around 6 to 8.

21. A method for making a nanoliposome ophthalmic suspension base, comprising:
a) preparing a mixture of a polysorbate surfactant and a cation selected from the group consisting of a cetylpyridinium halide, a cetrimonium halide, a cetalkonium halide, or mixtures thereof;
b) mixing lecithin and sterile water for injection;
c) preparing a combined mixture of the polysorbate surfactant and cation mixture and the lecithin and sterile water mixture;
d) processing the combined mixture through a microfluidizer to form a nanoliposome; and
e) incorporating the nanoliposome into a cellulose ether.

22. The method according to claim 21, further comprising adding a pharmaceutically active agent to the nanoliposome.

23. The method according to claim 21, further comprising adjusting the pH of the nanoliposome ophthalmic suspension base around 6 to 8.
